(19) United States
(12) Patent Application Publication
(10) Pub. No.: US 2017/0065298 A1
(43) Pub. Date: Mar. 9, 2017

(54) Hand-Held Fetal Head Elevator

(71) Applicants: Adam Harris, Dallas, TX (US); Anu Alturu, Kingwood, TX (US); Sandeep Ganji, Dallas, TX (US); Elizabeth Carstens, Tulsa, OK (US); Daniel Walk, Dallas, TX (US)

(72) Inventors: Adam Harris, Dallas, TX (US); Anu Alturu, Kingwood, TX (US); Sandeep Ganji, Dallas, TX (US); Elizabeth Carstens, Tulsa, OK (US); Daniel Walk, Dallas, TX (US)

(21) Appl. No.: 15/257,500

(22) Filed: Sep. 6, 2016

Related U.S. Application Data
(60) Provisional application No. 62/213,975, filed on Sep. 3, 2015.

(51) Int. Cl. A61B 17/44; A61B 5/00; A61B 5/03; A61B 5/145; A61B 5/024; A61B 8/00

(52) U.S. Cl. CPC A61B 17/442; A61B 5/02411; A61B 8/4444; A61B 5/035; A61B 5/14539; A61B 5/4362; A61B 2017/445

(57) ABSTRACT

A surgical device, and related method to aid in delivery by vaginal or cesarean section delivery to dislodge the fetal head when it has become wedged in the birth canal is provided. The device has a body and a handle for an operator to hold and brace against the mother, and a head plate with an inflatable bladder for gently applying force to the fetal head. The head plate is inserted into the birth canal and the bladder is filled with air or biocompatible fluid. The combination of insertion and inflation gently elevates the fetal head out of the birth canal into the pelvis to facilitate repositioning or extraction of the fetus. The device may have an adjustable neck to accommodate different anatomies and scenarios. The device may have sensors to monitor the health of both the neonate and the mother and to aid in the operation of the device.

### CROSS-REFERENCE TO RELATED APPLICATIONS

This application claims benefit under 35 U.S.C. §119 and incorporates by reference United States Provisional patent application for FETAL HEAD ELEVATOR by inventors Adam Harris, Anu Atluru, Sandeep Ganji, Elizabeth Carstens, and Daniel Walk, filed electronically with the USPTO on Sep. 3, 2016 with Ser. No. 62/213,975, EFS ID 23400158, confirmation number 3494, docket ASAFC.0101.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF INVENTION

1. Field of Invention

The present invention generally relates to apparatuses, and methods that aid in the delivery of a neonate during childbirth, and specifically to apparatuses, and methods associated with assisting delivery of a neonate by minimizing complications associated with, including but not limited to, the orientation and advancement of the neonate during childbirth.

2. Description of Related Art Including Information Disclosed Under 37 C.F.R. 1.97 and 1.98

The safe delivery of a neonate with as few complications for mother and child as possible is the goal of every labor. Ideally, this would be a quick process as prolonged labor can be physically stressful and dangerous with possible complications as severe as death for both the mother and child. Therefore every attempt is made to deliver the child quickly and the failure of labor to move steadily towards a delivery is often a reason to perform a Caesarian section.

One of the most common reasons for labor to stop progressing is that the neonate's head is not properly aligned within the mother's birth canal. Not only must the neonate be head down in cephalic presentation, but it must be rotated to align with the mother's anatomy to pass through the narrow birth canal. Both the neonate's head and the mother's pelvis are not circular in cross section but rather oval, and thus they must align to allow the head to pass through. Lack of alignment can cause the neonate's head to become stuck and can obstruct the delivery process altogether.

When labor fails to progress, the physician uses all the tools at his disposal to determine the cause and correct it. If the woman is tired, he may consider giving her fluids or medication to increase uterine contractions. He may alternatively try to feel for the neonate's head. If he believes it is out of alignment, he may have the woman assume several different positions for a period of time in attempt to reorient the child within the birth canal. The physician may attempt to pull the neonate through the birth canal with forceps or a vacuum delivery device. However, there is no current best practice that can be done to directly intervene and fix an alignment problem.

If the alignment problem is not quickly resolved, a caesarian delivery may be necessary to protect both the mother and a neonate. In Cesarean section deliveries, the neonate is delivered through an incision first in the abdomen and then in the uterus. An upward pulling force is required in order to pull the neonate through the incisions and out of the mother. Most often the force is provided by the doctor's hands directly pulling the neonate through the abdominal incision. And in the majority of cases, this is enough to deliver the neonate.

Cesarean section deliveries are performed in about a third of the births in the United States, specifically 32.8% in 2012. Cesarean section rates have increased from 22.3% in 1992, paralleled by increasing rates of primary cesarean delivery (32.1% in 2012) and a decrease in vaginal birth after C-section (10.2% in 2012). Accordingly, approximately 90% of women who undergo a primary C-section will have a repeat C-section in subsequent pregnancies. The rising prevalence of advanced maternal age, obesity and other comorbidities and use of assisted reproductive technology are factors in underlying C-section rates. C-section rates vary internationally but are generally increasing globally as well.

There are particular cases when the forces of labor have pushed the neonate's head so that it is wedged in the birth canal such that the physician cannot complete maneuvers to slide his hand under the neonate's head to lift it out of the incisions. This is called an impacted fetal head and is a significant medical complication of Cesarean delivery following a prolonged second stage (active contraction) of labor.

There are no proven methods of predicting fetal head impaction thus it commonly presents as prolonged second stage of labor with eventual arrest of labor. A longer duration of the second stage of labor is associated with adverse maternal outcomes, including higher rates of infection, trauma, and postpartum hemorrhage. Moreover, for each hour of the second stage, the chance for spontaneous vaginal delivery decreases progressively, leading to attempted operative delivery or cesarean section. Fetal head impaction itself and the currently required traumatic delivery can result in fetal complications including intracranial hemorrhage, skull and neck fractures, asphyxia, and increase incidence of neonatal intensive care unit admission.

Currently, to address an impacted fetal head, the physician has two techniques. He may either pull on the neonate's legs or ask an assistant to push on the crown of the neonate's head through the vaginal canal. These techniques both carry significant risk. Pulling on the neonate's feet can cause extension damage to the fetal neck and spinal cord. Applying force to the neonate's head while still in the womb involves using the tips of one's fingers or possibly a fist. The force is applied over a small area with a fairly rigid instrument. This push maneuver on the neonate's crown can cause intracranial bleeding, skull fractures, lower Apgar scores, and hematomas in the neonate and hemorrhage in the mother, typically due to the extension of the uterine incision into the adjacent uterine arteries as well as thromboembolism and infection. The potential for trauma makes this a risky maneuver and limits the number of applications where one could interact with the fetus.

FIG. 1 depicts a fetus's skull 100 at birth, showing the anterior and posterior fontanelles. A fontanelle, also called fontanel or soft spot, is an anatomical feature of a fetus's skull comprising of any of the soft membranous gaps (sutures) between the cranial bones that make up the calvaria of an fetus. Fontanelles allow for rapid stretching and deformation of the neurocranium as the brain expands faster than the surrounding bone can grow. Fontanelles also enable the bone plates of the skull to flex, during birth, allowing the fetus's head to pass through the birth canal. Even though the fontanelles allow for an fetus's head to flex to pass through the birth canal, the size of an fetus's skull will still cause damage to the mother's lower uterine structures as well as pelvic structures.

Several options exist that aim to alleviate impacted fetal head during vaginal delivery but their use is waning. Metallic fetal head elevators such as the Coyne spoon and the Murless head extractor, mechanistically similar to the “shoe horn,” assist in the extraction of the fetal had and are associated with significant maternal soft tissue trauma (e.g. severe perineal tears). Vacuum extractors including the Kiwi OmniCup and the Malmstrom metal cup conform to the fetal head and create negative pressure, assisting in maneuvering the fetal head vaginally. Vacuum extraction has been shown to be more effective than other vaginal delivery assist devices but carries increase risk of neonatescalp injury and cephalohematoma. Failed attempts at instrumental extraction, especially with vacuum extraction can counterproductively lodge the head deeper in the pelvis.

U.S. Patent Application Publication US 2008/0154284, entitled Device to Assist in Cesarean Section, by Rajiv Varma, provides a surgical device that allows for the easy delivery of the neonateduring Cesarian Section when the fetal head is deeply wedged in the female pelvic cavity. FIG. 2 depicts the use 200 of the surgical device as taught by Varma. This surgical device is placed inside the vagina below the neonate's head 210, and the device is held in place by the vagina. The surgical device is used as a jack to lift the fetal head while using the uterine walls to stabilize the device. The problem is that this device puts pressure on the uterine walls and on the birth canal for stabilization, which can be detrimental to the mother's health during labor. Moreover, to be fully deployed, the device must be installed very early in the labor process before significant effacement of the cervix and birth canal has occurred. This is well before the time that prolonged second stage and fetal impaction present, making the device fundamentally flawed in for its intended purpose.

U.S. Patent Application Publication US 2013/0158563, entitled Article and Method for Assisting Cesarean Section Delivery, by Diana Madden Adams, provides an elongated flexible member having a pocket centrally located to help a surgeon guide and dislodge the fetus's head. This method of dislodging the fetus's head during a cesarean section also puts pressure on the uterine walls and on the birth canal because the surgeon is stretching the uterine walls and the birth canal in order to dislodge the fetus's head.

U.S. Patent Application Publication US 2004/0018175, entitled Tube of Air Bag—Design on Glove, Forceps and Vacuum Use to Open the Birth Canal—Labor Delivery, by Bich Nguyen, provides a technique for stretching the birth canal and pulling the fetus's head through the birth canal using a vacuum. As mentioned previously, this method of delivery applies pressure to the uterine walls and birth canal by stretching out the uterine walls and birth canal, which may cause tears and bleeding.

U.S. Pat. No. 3,794,044, entitled Delivery Forceps by William Vennard and Arthur Decker, provides a method and apparatus for assisting delivery of a neonate during childbirth which includes surrounding the upper portion of the fetus's head with multi-pronged forceps and pulling the forceps to assist in delivery of the neonate from the uterus. A membrane also connects the multiple prongs of the forceps allowing a vacuum to be applied to secure the device to the fetus's head. However, this method and apparatus applies pressure on the fetus's skull at the tips of the prongs of the forceps, and the force of the prong tips on the skull during the pulling may cause damage to the fetal head.

Therefore these techniques and devices are limited in use and the amount of force that can be applied to the fetal head, and current best practice involves using these techniques simultaneously to quickly dislodge the fetal head because speed is of the essence in this procedure. While the American College of Obstetricians and Gynecologists and the American Academy of Pediatrics suggests that hospitals be able to perform a C-section within thirty minutes of recognition, fetal anoxia complications can begin within as few as five minutes. There is also an increased risk of severe hemorrhage, anesthetic complication or accidental injury to the fetus as time to delivery lengthens.

At this time, there is no device to assist with impacted fetal head without putting pressure on the uterine walls or the birth canal, and there is no device that can be used to directly interact with a misaligned neonate while still in the mother's uterus. A device that could assist with both of these conditions could both reduce the number of Caesarian deliveries and reduce the rate of negative outcomes of one of the highest risk complications of this major surgery.

### BRIEF SUMMARY OF THE INVENTION

Disclosed herein is an apparatus and related method for the purpose of directly interacting with a neonate during children so as to assist in the delivery of the fetus.

In accordance with one embodiment of the present invention, an apparatus and method for directly interacting with a neonate during childbirth so as to assist in the delivery of the neonate are provided which substantially eliminates or reduces disadvantages associated with previous systems, methods, and techniques.

In accordance with one embodiment of the present invention, an apparatus for aiding delivery of a neonate during childbirth is provided. The apparatus comprises a head plate; an operator handle; and a body connecting the head plate and the operator handle. The apparatus may optionally comprise a maternal stabilization plate. The head plate of the apparatus may have a disposable and replaceable surface. The head plate may also have an inflatable bladder, which may be manually or automatically inflated. The head plate may be attached to the rest of the apparatus via an adjustable neck piece. The apparatus may also have hollow channels, for fluid to pass through to a vaginal opening, and sensors. At least one of the sensors may be an ultrasound probe, a fetal heart rate monitor, a pressure sensor, or a pH sensor. Optionally, the apparatus may comprise a sensor display for the sensors of the apparatus.

In accordance with another embodiment of the present invention, a method for aiding delivery of a neonate during childbirth, wherein the head of the neonate is lodged in a birth canal is provided. The method comprises inserting a head plate into the birth canal until a bladder attached to the head plate touches the head of the fetus; inflating the bladder; and adjusting a position of the head of the neonate so that an operator may apply force to a handle attached to a body, wherein the body is attached to the head plate, and the force transfers to the head plate to push the neonate back in the birth canal. Bladder inflation may be done manually or automatically. The method may further comprise injecting fluid through a fluid port into the birth canal, inserting a set of sensors to monitor the fetus's health, and inserting a set of sensors to monitor a mother's health.

At least one advantage attributable to novel aspects of the present disclosure is distributing the amount of force/pressure used against the fetus's head.

### BRIEF DESCRIPTION OF THE DRAWINGS

The present invention will be more fully understood by reference to the following detailed description of the preferred embodiments of the present invention when read in conjunction with the accompanying drawings, wherein:

FIG. 1 depicts a fetus's skull at birth, showing the anterior and posterior fontanelles.

FIG. 2 depicts the use of the surgical device as taught by U.S. Patent Application Publication US 2008/0154284, entitled Device to Assist in Cesarean Section, by Rajiv Varma.

FIG. 3A depicts a perspective view of an exemplary embodiment of an apparatus that enables direct interaction with a neonate through the birth canal.

FIG. 3B depict a side view of an exemplary embodiment of the present invention including an inflatable bladder attached to the head plate.

FIG. 3C depict a side view of an exemplary embodiment of the present invention including an inflated inflatable bladder attached to the head plate.

FIG. 4A depicts a side view of the inflatable bladder deflated and inflated. The inflatable bladder is connected to a connector.

FIG. 4B depicts a front view of the inflatable bladder inflated.

FIG. 5 depicts another embodiment of the present invention with a tamponade bladder.

FIG. 6 depicts a side view of an exemplary embodiment of the present invention with two ports leading to the head plate.

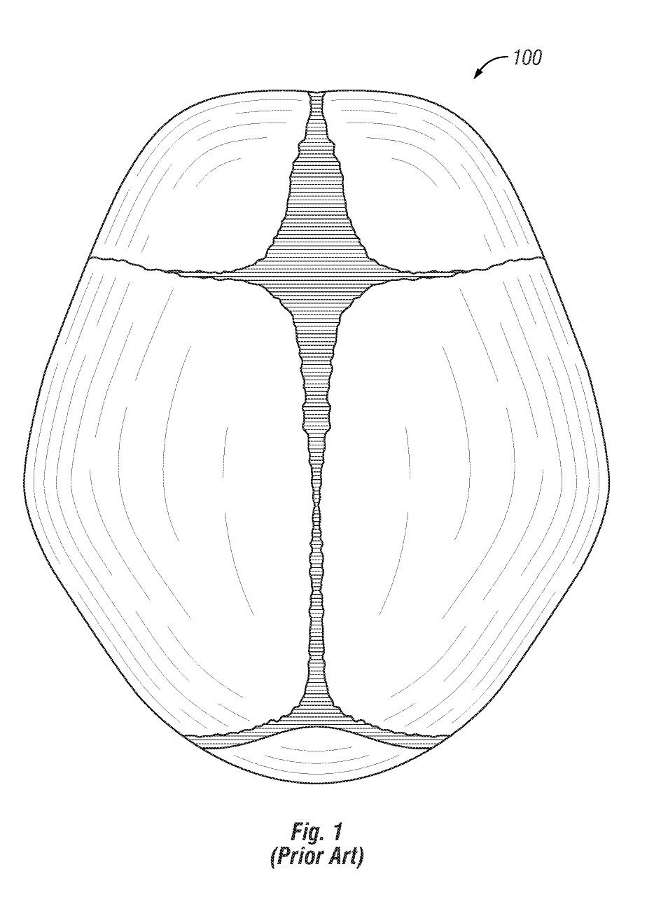

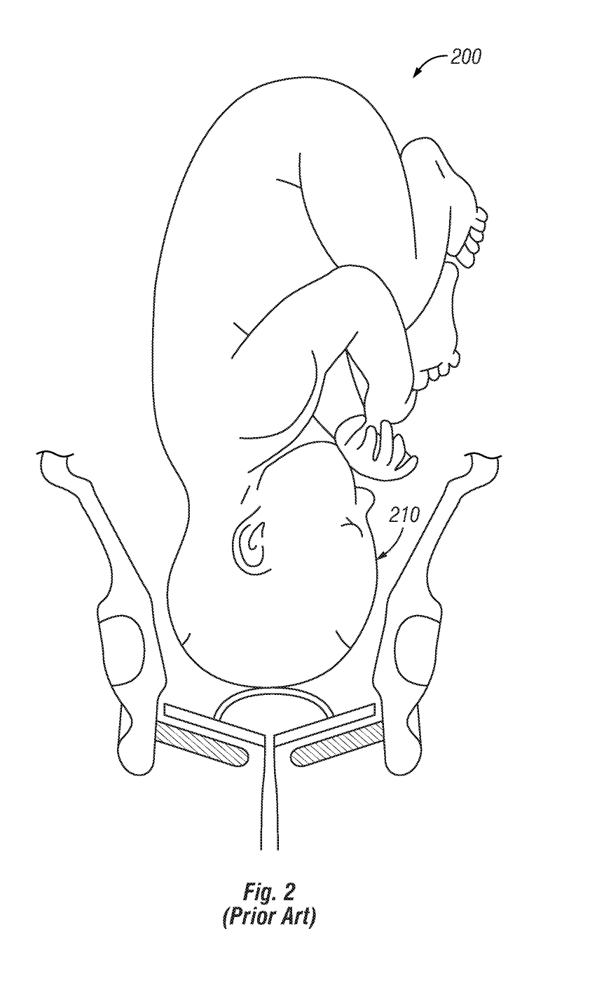

The above figures are provided for the purpose of illustration and description only, and are not intended to define the limits of the disclosed invention. Use of the same reference number in multiple figures is intended to designate the same or similar parts. Furthermore, when the terms “top,” “bottom,” “first,” “second,” “upper,” “lower,” “height,” “width,” “length,” “end,” “side,” “horizontal,” “vertical,” and similar terms are used herein, it should be understood that these terms have reference only to the structure shown in the drawing and are utilized only to facilitate describing the particular embodiment. The extension of the figures with respect to number, position, relationship, and dimensions of the parts to form the preferred embodiment will be explained or will be within the skill of the art after the following teachings of the present invention have been read and understood

### DETAILED DESCRIPTION OF THE INVENTION

A portion of the disclosure of this patent document contains material which is subject to copyright protection. The copyright owner has no objection to the facsimile reproduction by anyone of the patent document or the patent disclosure, as it appears in the Patent and Trademark Office patent file or records.

While this invention is susceptible of embodiment in many different forms, there is shown in the drawings and will herein be described in detailed preferred embodiment of the invention with the understanding that the present disclosure is to be considered an exemplification of the principles of the invention to the embodiment illustrated. It is understood, therefore, that the terminology, examples, drawings, section, and embodiments are illustrative and not intended to limit the scope of the disclosure.

The numerous innovative teachings of the present application will be described with particular reference to the presently preferred embodiment, wherein these innovative teachings are advantageously applied to the particular problems of a FETAL HEAD ELEVATOR. However, it should be understand that this embodiment is only one example of many advantageous uses of the innovative teachings herein. In general, statements made in the specification of the present application do not necessarily limit any of the various claimed inventions. Moreover, some statements may apply to some inventive features but not to others.

The terms “newborn,” “child,” “fetus,” “foetus,” “neonate,” “baby”, “infant” may be used interchangeably in the description that follows. The terms “pressure” and “force” may be used interchangeably in the description that follows.

The term “computer processing device” or computing device means any electrical device capable or accepting stored program instructions from a computer readable medium and processing those program instructions to perform a defined task. Such devices include, but are not limited to, a mainframe, workstation, desktop, laptop, notebook, or tablet computer, a database server, web server, or the like. One of ordinary skill in the art will appreciate that the construction, choice of programming language, programming, operation, and functionality of such computer processing devices is well known, rendering further description of such devices unnecessary in this regard.

The invention is designed to lift a fetus's head out of the birth canal without applying pressure to the uterine walls and the birth canal, and back into the pelvis where it can more freely move within the uterus. The present invention may be used during any part of the delivery process where direct interaction with the neonate's head through the birth canal is desired or where moving or reorienting the neonate's head relative to the birth canal is desired.

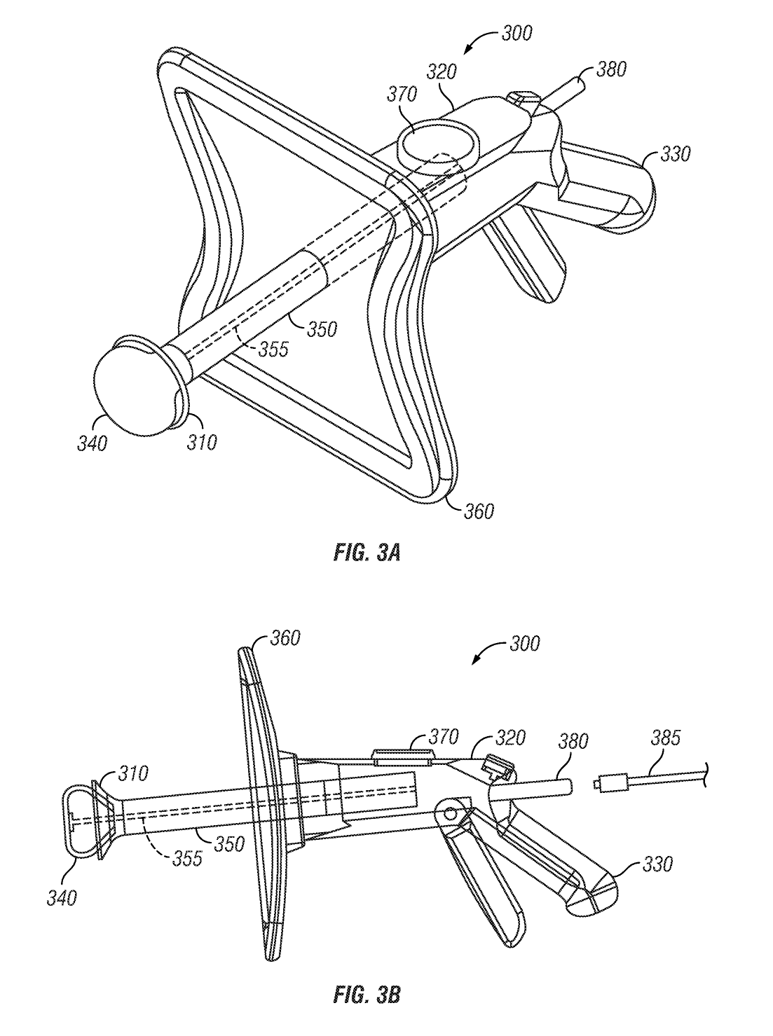

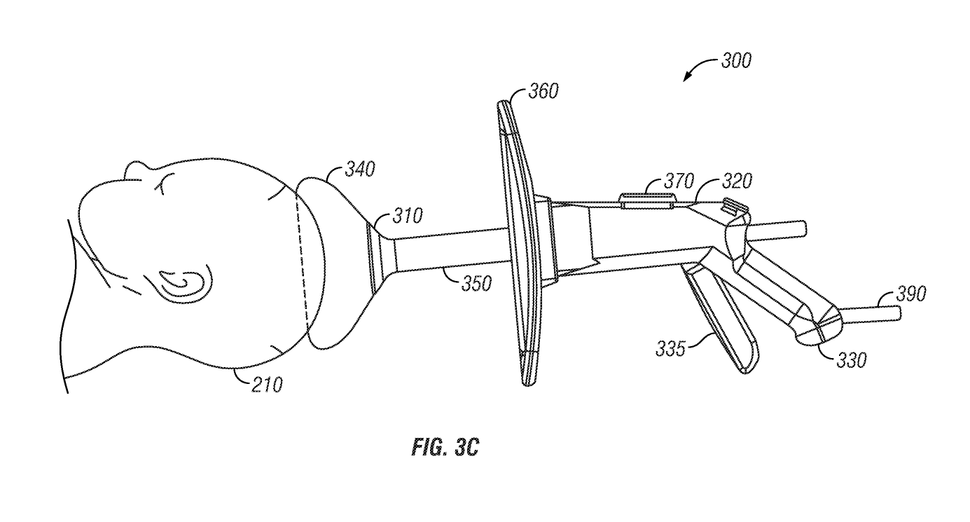

FIGS. 3A, 3B, and 3C are different views of the fetal head elevator device 300. FIG. 3A depicts a perspective view of an exemplary embodiment of an apparatus 300 that enables direct interaction with a neonate through the birth canal. FIG. 3B depict a side view of an exemplary embodiment of the present invention including an inflatable bladder 340 attached to the head plate. A head plate 310 is connected to a body 320 via a neck 350. The body 320 is also connected to a handle 330 for an operator of the fetal head elevator device 300. An operator holds the fetal head elevator 300 with the handle 330 and inserts the head plate 310 into the vaginal opening to rest against the neonate's head 210 and can then apply force through the handle 330, which remains outside the mother's body. Pressure applied to the handle 330 is transmitted through the body 320 to the fetus's head 210 which elevates it away from the birth canal so that it can be more easily manipulated within the uterus. The present invention is designed to be used during the delivery process whenever readjusting the location or orientation of the neonate's head 210 is desired.

The head plate 310 is inserted vaginally during labor so that the head plate 310 touches the neonate's head. The head plate 310 may be constructed from rigid material, such as plastic or metal. The head plate 310 distributes the force applied to the neonate's head 210 over a much greater surface area. The head plate 310 is designed to spread the transmitted pressure from the handle 330 over a greater surface area of the fetus's head 210. The head plate 310 may employ a padding or bladder or similar structure to further reduce high pressure to the fetus's head 210.

In alternative embodiments, the head plate 310 may be made of foam or a soft jelly-like material so as to soften and lubricate the impact or force/pressure applied to the fetus's head 210 when the head plate 310 contacts the fetus's head 210.

The handle 330 is connected to the body of the fetal head elevator device 300, and remains outside the mother's body. The operator transmits a force to the handle 330, which in turn transmits to the body 320 and to the head plate 310. The handle 330 may be of any cross-sectional shape, and may be constructed of any rigid material, such as plastic or metal. The handle 330 may have an ergonomic shape to find into the hand of the operator. Alternatively, the handle 330 may be shaped to provide a housing structure for other medical instruments, such that the present invention can be coupled with other medical devices. The handle 330 may comprise a mounting bracket to allow attachment to a mechanical arm, operating table, or other fixed surface.

The handle 330 may have controls for managing pressure or for manipulating the body or the head plate.

In one embodiment of the present invention, the neck 350 is adjustable. The adjustable neck connects the head plate 310 to the body 320 of the fetal head elevator device 300. The adjustable neck 350 adjusts in length and in angle (with respect to the birth canal) to accommodate the different anatomies of different mothers and the different locations of the fetal head 210. Alternatively, the adjustable neck 350 may contort in shape to provide the adjustable angle needed to reach the fetal head. The adjustable neck 350 may also be fixed into position in order to stabilize the device 300, so that when force is applied from the operator and the handle 330, the adjustable neck 350 remains fixed and can transfer the force to the head plate 310 and the fetus's head 210.

A hollow channel 355 may pass through the neck 350 and through the body 320 and connect to a port 380 at the end of the body 320. The hollow channel 355 permits sensors 385 to pass through to the birth canal. The type of sensors 385 that pass through this hollow channel 355 is not limited by the size or shape of the hollow channel 355. Sensors 385 to be used with the fetal head elevator device 300 include but are not limited to ultrasound probes, video cameras, light probes, fiber optic scope, heart rate monitors, or related devices. The sensors 385 monitor the fetal anatomy, assess the fetal well-being, or aid in the manipulation of the fetus's head 210. The sensors 385 may also monitor and assess the mother's well-being. Because some sensors 385 need to remain in a particular position or shape, the hollow channel 355 may be appropriately shaped to receive the sensors.

The sensors 385 in this embodiment of the present invention may trigger an alert in the fetal head elevator device 300. The fetal head elevator device 300 may contain a computing device to show the readings from the sensors 385. Once the sensors 385 detect a certain threshold has been met or surpassed, then the fetal elevator device 300 alerts the operator so that the operator may take appropriate actions for the well-being of the mother and the fetus.

The information received from the sensors 385 may also be displayed on a display 370 on the body 320. The sensors 385 may be used to measure pH, fetal heart rate, pressure, oxygen saturation, a video image of the fetal head 210, and display the results on the display 370. Alternatively, this display 370 could be a pressure gauge. The display/gauge 370 reads the pressure inside of the bladder 340, so that an operator will know the force of the bladder 340 against the fetus's head 210. The display/gauge 370 may also read the pressure or force asserted against the fetus's head 210.

In yet another embodiment of the present invention, the fetal head elevator device 300 is connected to a maternal stabilization plate 360 designed to stabilize the device 300 during the insertion, adjustment of the head plate 310, or the application of force/pressure to the fetus's head 210. The shape of the maternal stabilization plate 360 may be shaped to accommodate the female anatomy, such as legs, buttocks, or other female anatomic structures. The maternal stabilization plate 360 may contact the mother's perineum in order to further stabilize the fetal head elevator device 300.

The maternal stabilization plate 360 may be transparent or translucent and constructed from plastic, metal, or any other rigid material. The plate 360 may have lights that project into the birth canal to aid visualization. The plate 360 can be fixed to the fetal head elevator device 300, or it can moved into a position (via sliding, spinning, pivoting, etc.) relative to the body of the fetal head elevator device 300 and locked into place.

As mentioned previously, the head plate 310 may be attached to a bladder 340, as shown in FIGS. 3A and 3B, and the bladder 340 may be inflated. The inflatable bladder 340 modulates the force or pressure applied to the fetus's head. Alternatively, the inflatable bladder 340 applies the force or pressure transmitted from the handle in a more measured or controlled fashion. In alternate embodiments, padding may instead be attached to the head plate 310 in order to reduce the amount pressure applied to the fetus's head 210.

FIG. 3C depict a side view of an exemplary embodiment of the present invention including an inflated inflatable bladder 340 attached to the head plate 310. FIGS. 3A and 3B depicts the present invention with the inflatable bladder 340 deflated, and FIG. 3C depicts the inflatable bladder 340 inflated. The inflatable bladder 340 may be constructed from plastic, elastic, or any other flexible material resistant to punctures. The inflatable bladder 340 may be able to stretch during the inflation process. The inflatable bladder 340 may be inflated with air, liquid, or any other biocompatible substance. The bladder 340 may accommodate an internal rigid support structure to maintain an effective form. The structure of the bladder 340 may conform to the neonate's head 210; alternatively, the bladder 340 may have a shape to accommodate the neonate's head 210 and distribute the force applied from the handle 330 and operator over a wider surface area.

In order to inflate the inflatable bladder 340, the handle 330 of the fetal elevator device 300 may connect the bladder 340 to a fluid or air reservoir through the body 320 at a fluid port 390, and the handle 330 has a trigger for a manual pump 335, and a pumping tube may deliver the substance used to inflate the inflatable bladder 340. The body 320 may connect the pumping tube to the bladder 340 bypassing the handle, allowing for easier handling by the operator. The manual pump 335 delivers the substance through a hollow channel, similar to the hollow channel 355, to the inflatable bladder 340. Alternatively, an automatic pump, syringe, or pressure system may be used to inflate the inflatable bladder 340. One of ordinary skill in the art would understand how to inflate the inflatable bladder 340.

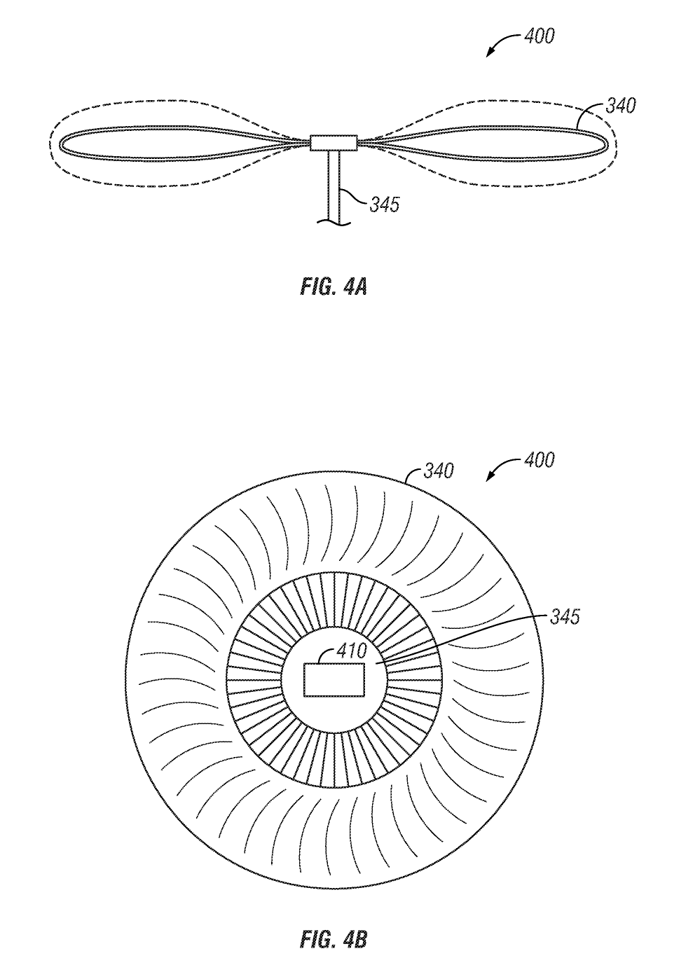

FIG. 4A depicts a side view 400 of the inflatable bladder deflated and inflated. The inflatable bladder is connected to a connector 345. The connector 345 connects the bladder 340 to the head plate 310 and to the neck 350 of the fetal elevator device 300. The connector 345 is also connected to a fluid or air reservoir through either a hollow channel or a pumping tube.

FIG. 4B depicts a front view of the inflatable bladder 340 inflated. The inflated inflatable bladder 340 may comprise any cross-sectional shape; however, it is preferred for the inflatable bladder 340 to have a shape to accommodate the neonate's head 210 so that when force/pressure is applied to the neonate's head 210, the force is distributed across the area of the inflatable bladder 340 in contact with the neonate's head 210. Alternatively, the neonate's head 210 may contact the head plate 310, and the inflatable bladder 340 may assist the transmission of force from the device 300 to the neonate's head 210. FIG. 4B illustrates a front view of a conical or semi-spherical structure for the inflatable bladder 340. When the head plate 310 and the deflated inflatable bladder 340 is inserted vaginally, the operator of the fetal head elevator device 300 applies force on the handle 330 while the inflatable bladder 340 begins to inflate. The inflatable bladder 340 helps lift the neonate's head 210 out of the birth canal, and allows either repositioning during labor or extraction during delivery by caesarian section.

A sensor pad 410 may be located on the connector 345 at the apex or center of the bladder 340. The sensor pad 410 may aggregate the sensors 385 for easier passage through the hollow channel 355. The sensor pad 410 may include a light source for easier visualization for the operator during the insertion of the head plate 310 and the inflatable bladder 340 into the birth canal. Sensors could include a pH sensor, a heart rate sensor, a pressure sensor, or an ultrasound probe. This list is not exhaustive of the sensors that could be included.

The connector 345 may have port outlets for fluids, such as medications or lubrication, so that the fluids can assist in the insertion of the fetal head elevator device 300 into the birth canal.

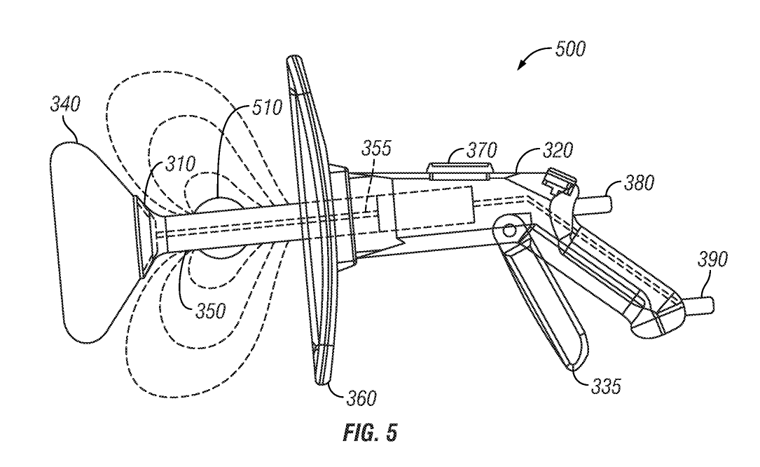

FIG. 5 depicts another embodiment of the present invention with a tamponade bladder 510. The tamponade bladder 510 starts off as another deflated bladder attached to and surrounding the neck of the fetal head elevator. The tamponade bladder 510, like the inflatable bladder 340, may be constructed from plastic, elastic, or any other flexible material resistant to punctures. The tamponade bladder 510 inflates, as shown in FIG. 5, to apply pressure against the birth canal in order to staunch bleeding from tears of the birth canal during childbirth. The use of the tamponade bladder 510 differs from the current state of the art because the tamponade bladder 510 is not used to stabilize the device 300 against the mother while in use. The tamponade bladder 510 staunches bleeding by creating a higher pressure environment, and the fetal head elevator device 300 may have a telescoping neck so to continue adjusting the fetus's position in the birth canal.

Because the tamponade bladder 510 when in use is stationary, the neck 350 of the fetal head elevator device 300 may telescope and allow the operator to apply pressure to the fetal head 210 via extending the length of the neck 350 without moving the tamponade bladder 510 once the tamponade bladder 510 is in use.

In another embodiment of the present invention, the tamponade bladder 510 may be covered with an elastic absorbent material. The material stretches to accommodate the inflation of the tamponade bladder 510, and during the inflation process, the material absorbs blood from tears in the birth canal. With the material absorbing the blood, an operator using a camera sensor can more easily operate the fetal head elevator device 300.

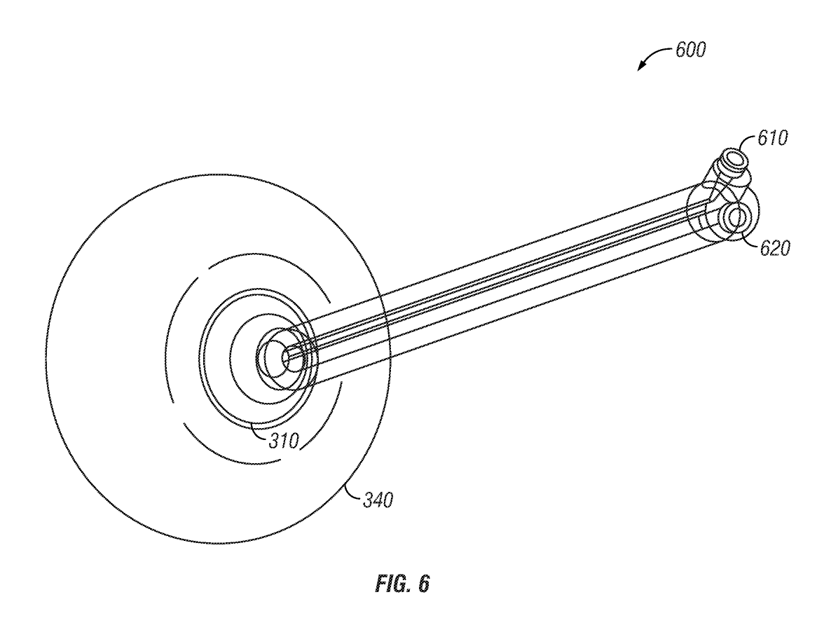

FIG. 6 depicts a side view of an exemplary embodiment 600 of the present invention with two ports leading to the head plate. The first port 610 is connected a first hollow channel which is connected to the sensor port, and the second port 620 is connected to a second hollow channel which is connected to the fluid port.

Alternatively, the first port 610 may be connected to a lubrication or medication reservoir. The device 300 has a hollow channel or tube that allows the passage of a fluid or a similar substance into the birth canal when the head plate is inserted vaginally. Port outlets may be located on the connector at one end of the fetal head elevator, and is connected to the end of the hollow channel to the other port at the other end of the device 300. The fluid port remains outside of the mother's body so that a fluid tube may be attached to the fluid port, and the port outlets may be inside of the mother's body and allow access to the birth canal. The hollow channel in this exemplary embodiment may be distinct from the hollow channel that transmits fluid or air to the inflatable bladder, or the hollow channel used for sensors. Alternatively, the hollow channel or tube may travel along the outside of the body 320 instead of through the body 320. The operator attaches a fluid tube to the port outside of the mother's body and allows fluid to pass through the fluid tube into the fetal head elevator device 300. The fluid flows through the hollow channel and out of the port inside of the mother's body. The fluid may be a fluid that lubricates the birth canal, either allowing for easier delivery of the neonate through the birth canal or through caesarian section.

The invention may be embodied in other specific forms without departing from the spirit or essential characteristics thereof. The present embodiments are therefore to be considered in all respects as illustrative and not restrictive. Accordingly, the scope of the invention is established by the appended claims rather than by the foregoing description. All changes which come within the meaning and range of equivalency of the claims are therefore intended to be embraced therein. Further, the recitation of method steps does not denote a particular sequence for execution of the steps. Such method steps may therefore be performed in a sequence other than recited unless the particular claim expressly states otherwise.

1. An apparatus for aiding delivery of a neonate during childbirth, the apparatus comprising
a head plate;
an operator handle; and
a body connecting the head plate and the operator handle.

2. The apparatus of claim 1, further comprising a maternal stabilization plate.

3. The apparatus of claim 1, wherein the head plate comprises a disposable and replaceable surface.

4. The apparatus of claim 1, wherein the head plate comprises an inflatable bladder

5. The apparatus of claim 4, wherein the inflatable bladder is manually inflated.

6. The apparatus of claim 4, wherein the inflatable bladder is automatically inflated.

7. The apparatus of claim 1, further comprising a set of hollow channels.

8. The apparatus of claim 7, wherein at least one of the set of hollow channels allows fluid to pass through to a vaginal opening.

9. The apparatus of claim 1, further comprising a set of sensors.

10. The apparatus of claim 9, wherein at least one of the set of sensors is an ultrasound probe.

11. The apparatus of claim 9, wherein at least one of the set of sensors monitors fetal heart rate.

12. The apparatus of claim 9, wherein at least one of the set of sensors detects the pressure of the head plate.

13. The apparatus of claim 9, wherein at least one of the set of sensors detects pH level.

14. The apparatus of claim 1, wherein the head plate is connected to the body via an adjustable neck piece.

15. The apparatus of claim 1, further comprising a sensor display.

16. A method for aiding delivery of a neonate during childbirth, wherein the head of the neonate is lodged in a birth canal, comprising:
inserting a head plate into the birth canal until a bladder attached to the head plate touches the head of the fetus;
inflating the bladder; and
adjusting a position of the head of the neonate so that an operator may apply force to a handle attached to a body, wherein the body is attached to the head plate, and the force transfers to the head plate to push the neonate back in the birth canal.

17. The method of claim 16, wherein inflating the bladder comprising manually inflation.

18. The method of claim 16, further comprising injecting fluid through a fluid port into the birth canal.

19. The method of claim 16, further comprising inserting a set of sensors to monitor the fetus's health.

20. The method of claim 16, further comprising inserting a set of sensors to monitor a mother's health.

21. A method to assist in the adjustment of the trajectory of fetal decent by correcting the positioning of the fetal skull relative to the birth canal, comprising:
inserting a head plate into the birth canal until a bladder attached to the head plate touches the head of the fetus;
inflating the bladder; and
adjusting a position of the head of the neonate so that an operator may apply force to a handle attached to a body, wherein the body is attached to the head plate, and the force transfers to the head plate to push the neonate back in the birth canal.
